(19) United States
(12) Patent Application Publication
(10) Pub. No.: US 2006/0115841 A1
(43) Pub. Date: Jun. 1, 2006

(54) Method and system for elucidating the primary structure of biopolymers

(75) Inventors: Martin Bluggel, Dortmund (DE); Daniel Chamrad, Bochum (DE); Helmut E. Meyer, Recklinghausen (DE)

Correspondence Address: CONNOLLY BOVE LODGE & HUTZ, LLP, P O BOX 2207, WILMINGTON, DE, 19899, US

(73) Assignee: PROTAGEN AG, Dortmund (DE)

(21) Appl. No.: 11/254,168

(22) Filed: Oct. 19, 2005

(30) Foreign Application Priority Data
Oct. 20, 2004 (DE) 102004051016.4-52

(51) Int. Cl. C12Q 1/68; G06F 19/00; G01N 33/00

(52) U.S. Cl. 435/6; 436/86; 702/19; 702/20

(57) ABSTRACT

The present invention relates to a method for elucidating the primary structure of biopolymers, in which a biopolymer to be investigated is cleaved into fragments and, after that, subjected to a mass spectrometric analysis (20) resulting in mass spectra being obtained, and in which known algorithms, are used for a first sequence analysis (30) of the fragments in order to determine a primary structure of the biopolymer using the mass spectra.

The mass spectra are classified in dependence on results of the first sequence analysis (30), resulting in at least one first spectrum class, to which a known biopolymer can be assigned, and one second spectrum class, to which no known biopolymer can be assigned, being obtained. A further analysis (50) of mass spectra of the second spectrum class is carried out in dependence on the known biopolymer.

The present invention relates to a method for elucidating the primary structure of biopolymers, in which a biopolymer to be investigated is cleaved into fragments and, after that, subjected to a mass spectrometric analysis, resulting in mass spectra being obtained, and in which known algorithms are used for a first sequence analysis of the fragments in order to determine a primary structure for the biopolymer using the mass spectra.

The present invention also relates to a system for elucidating the primary structure of biopolymers.

The primary structure of biopolymers is understood as meaning the chemical structure, in particular an appurtenant sequence of the amino acids and their modifications such as posttranslational modifications or chemical modifications.

Within the context of this invention, therefore, a biopolymer is understood as meaning a modified or unmodified polypeptide containing at least one peptide bond and, where appropriate, nonprotein moieties such as lip(o)ids, carbohydrates or other organic moieties and/or inorganic moieties such as metals.

Elucidation of the primary structure is also understood here as meaning findings with regard to errors/divergences from/in relation to available sequence databases and modification databases and with regard to single amino acid polymorphisms (SAPs).

The primary structure is usually elucidated using mass spectrometric data. These mass spectrometric data are obtained by measurement using a variety of known mass spectrometric methods.

In mass spectrometry (MS in brief), methods such as electrospray MS (ESI MS) and various methods of laser desorption such as MALDI MS are particularly suitable for biopolymers (see, in a general manner, Budzikiewicz, Massenspektrometrie [mass spectrometry], Weinheim (1998)).

In the subsequent description, the term mass spectrometric data is understood, in particular, as meaning information with regard to the molecular weight (or m/z value) of biopolymers or parts (fragments) thereof which are obtained by specifically cleaving one or more biopolymers. Without restricting the generality, the term mass spectrum is also used for designating mass spectrometric data in that which follows.

In addition to this, the biopolymers can be modified specifically or unspecifically prior to cleavage and the cleavage itself can likewise be carried out specifically, i.e. at defined amino acids, or else unspecifically, i.e. independently of particular amino acids.

Posttranslational modifications, which are extremely important effectors of the physiological protein function and whose elucidation is also to be improved by the method according to the invention, constitute an important example of biopolymer modifications.

The mass spectrometric data are normally evaluated using bioinformatic analyses, where appropriate using a sequence database of known biopolymers, and, depending on the algorithm employed or depending on the bioinformatic analysis employed, the primary structure of the biopolymers, or of the fragments of the biopolymers, can be inferred from, for example, a comparison of the mass spectrometric data, which are obtained by measurement, and the data from the database.

Sequence databases contain either amino acid sequences of biopolymers or what are termed genomic sequences, from which the amino acid sequences can be deduced.

In the case of the known methods for elucidating the primary structure of biopolymers, the information which is obtained using clarified mass spectra of analyzed biopolymers is as a rule incomplete. As a rule, the analyzed mass spectra can only be assigned to a constituent sequence of a known biopolymer.

Furthermore, the situation can arise, when elucidating the primary structure of a biopolymer, that particular mass spectrometric data or mass spectra cannot be assigned to any known biopolymer, such that it is only partially possible, or not possible at all, to elucidate the primary structure of an investigated biopolymer.

It is therefore the object of the present invention to improve a generic method or system to the effect that the significance of the results of the elucidation of the primary structure is increased, the elucidation is completed and the method is at the same time simplified.

In the case of the described method, this object is achieved, in accordance with the invention, by the mass spectra being classified in dependence on results of the first sequence analysis, as a result of which at least one first spectrum class, to which, a known biopolymer can be assigned, and one second spectrum class, to which no known biopolymer can be assigned, are obtained, and by a further analysis of mass spectra of the second spectrum class being carried out in dependence on the known biopolymer.

In the context of the present invention, the known biopolymer is understood as meaning a biopolymer or an amino acid sequence which is assumed to be suitable for elucidating, for example, the mass spectra of the second spectrum class. That is, if a sufficiently good agreement can be established between the mass spectra which are obtained and a biopolymer which is obtained from a database, for example, the biopolymer from the database is used as a known biopolymer within the meaning of the invention. However, it is also possible to use only a particular part of this biopolymer, which is obtained from the database, as a known biopolymer for the method according to the invention. It is furthermore possible to use any other arbitrary amino acid sequence as a known biopolymer.

According to an advantageous embodiment of the present invention, peptides are obtained, as fragments of the biopolymer, when the biopolymer to be investigated is cleaved. The cleavage of the biopolymer to be investigated into peptides is carried out using known methods, for example by means of a so-called specific proteolysis. The enzyme trypsin, which cleaves on the C-terminal side of the amino acids arginine (R) and lysine (K), is frequently used for this purpose.

According to another very advantageous embodiment of the invention, peptide fragments are obtained, as fragments of the biopolymer, when the biopolymer to be investigated is cleaved. These peptide fragments are obtained from the peptides, which are obtained, for example, in the above-described manner, using techniques such as PSD (post source decay) or CID (collision-induced decay).

Relevant mass spectrometric data, which are included in the first sequence analysis in the form of mass spectra, are obtained, by means of mass spectrometric analyses, from both the peptides and the peptide fragments.

According to an advantageous embodiment of the present invention, the known algorithms used for the first sequence analysis are a peptide mass fingerprint (PMF) algorithm and/or a peptide fragmentation fingerprint (PFF) algorithm and/or algorithms from the family of the de-novo sequencing algorithms and/or PTM prediction algorithms and/or comparable algorithms.

The PMF algorithm makes it possible to elucidate the primary structure of a polypeptide on the basis of assigning a measured mass spectrum to an entry in a sequence database. The cleaving, by the PMF algorithm, of the sequences in the database into peptides with the same specificity as the analyzed biopolymer was previously cleaved into peptides results in a large number of peptide sequences from which the PMF algorithm can generate a theoretical mass spectrum for each entry in the sequence database.

By comparing measured mass spectra with the theoretically determined mass spectra, it is possible to give each database entry a weighting figure which is based on the result of the comparison and which reflects the degree of similarity between the mass spectra which have been compared. In the most favorable case, the database entry with the highest weighting figure corresponds to the sequence of the analyzed biopolymer.

In analogy with the PMF algorithm, the PFF algorithm also uses sequence databases. In this case, however, theoretical fragmentation spectra of peptides from the database are generated and compared with measured fragmentation spectra, from which comparison a database entry is once again identified by assessing the similarity.

The class of de-novo sequencing algorithms directly extracts information with regard to the primary structure of the analyzed biopolymer from fragmentation spectra of peptides, which spectra are obtained by measurement when analysing biopolymers. In contrast to the PMF and PFF algorithms, the de-novo sequencing algorithms do not use any sequence databases.

Another very advantageous embodiment of the method according to the invention is characterized by the fact that the further analysis exhibits the following steps:
modifying the known biopolymer in accordance with a modification rule which can be preset in order to obtain a modified biopolymer,
cleaving the modified biopolymer into fragments, preferably in accordance with a cleavage rule which can be preset,
forming theoretical mass spectra in dependence on the fragments which are obtained in connection with the cleaving of the modified biopolymer,
comparing the theoretical mass spectra with the mass spectra of the fragments of the second spectrum class.

This method variant according to the invention is based on the assumption that the mass spectra which it has not previously been possible to elucidate and which, for example, belong to the second spectrum class are derived from a biopolymer which only differs partially from the known biopolymer on account of a modification or that the unclarified mass spectra or the appurtenant fragments are obtained from an unexpected cleavage of the known biopolymer.

For this, the known biopolymer, which was ascertained in connection with the first sequence analysis, is used, in accordance with the invention, as the starting point for the subsequent analysis. The known biopolymer is then modified using freely selectable modification or cleavage rules.

After the modified biopolymer has been cleaved into fragments, which can in turn be peptides or peptide fragments, a mass spectrometric analysis, which leads to mass spectra which belong to the fragments, is then carried out.

The steps of the modification, the cleavage and the mass spectrometric analysis are, taking the known biopolymer as starting point, preferably performed theoretically, i.e. by means, for example, of a simulation, preferably using a suitable computer system.

Consequently, mass spectra, which are also termed theoretical mass spectra, are ipso facto obtained from the simulation in connection with the mass spectrometric analysis in accordance with the above-described method variant.

These theoretical mass spectra are compared with the mass spectra which are assigned to the fragments of the second spectrum class. An agreement of the compared mass spectra confirms the assumption, on which this method variant according to the invention is based, that mass spectra which were not previously elucidated, i.e. by means of the sequence analysis, for example, are to be assigned to a biopolymer which can be derived from the known biopolymer.

The above-described assumption makes it possible to markedly reduce the number of biopolymers to be investigated for clarifying the origin of the mass spectra of the second spectrum class, specifically down to one or more known biopolymers in the above-described sense, thereby accelerating the method and improving the elucidation rate.

In accordance with another variant of the invention, the known biopolymer can also initially be cleaved into fragments, preferably in accordance with a cleavage rule which can be preset. Subsequently, the fragments which are obtained by the cleavage of the known biopolymer can be modified in accordance with a modification rule which can be preset. After that, it is possible to form theoretical mass spectra in dependence on the modified fragments, which mass spectra can then be compared with the mass spectra of the second spectrum class.

According to another method variant, the sequence of the steps of modifying and cleaving is generally arbitrary. It is also possible to carry out individual steps, or all steps, several times. This thereby makes it possible to use an embodiment of the method according to the invention to in all model several modifications and/or cleavages.

In addition to this, the invention provides, where appropriate, for the step of cleaving and/or modifying to be entirely dispensed with.

Another very advantageous method variant provides for using, for the modification, a modification rule by means of which it is possible to model a post-translational modification and/or an amino acid substitution and/or a sequence error and/or a transpeptidation and/or random, and/or other, modifications of the known biopolymer.

According to another variant of the method according to the invention, it is possible to use, for the cleavage, a cleavage rule by means of which specific and/or unspecific cleavages of the known biopolymer and/or of the modified biopolymer can, be modeled. In this connection, the cleavage rule is preferably determined from a cleavage database.

In the case of another very advantageous embodiment of the method according to the invention, the modification rule is formed in dependence on data from a modification database. It is also very advantageous to combine several modification rules with each other.

Another very advantageous embodiment of the method according to the invention envisages a combination of several known algorithms for the first sequence analysis or the subsequent analysis, with this thereby increasing the significance of results which are obtained in connection with the given analysis.

The choice, according to the invention, of the cleavage or modification rule(s) can be regarded as being the advancing of a hypothesis according to which previously unidentified peptide mass spectra or peptide fragment spectra ensue from the known biopolymer as a result of the selected modification(s) or cleavage(s). Such a hypothesis is also termed a primary structure hypothesis.

It is also very advantageous to advance multistep primary structure hypotheses, because these latter are suitable for simultaneously taking account of several modifications of the biopolymer.

Particularly advantageously, the primary structure hypotheses are advanced in dependence on fragments which are preferably from the second spectrum class. This makes it possible to carry out the further analysis of previously unidentified peptide mass spectra or peptide fragment spectra particularly efficiently.

In another advantageous embodiment of the method according to the invention, it is possible to employ known, preferably statistical optimization methods for selecting modification rules or for advancing the primary structure hypothesis(ses). It is particularly advantageous to use random walk methods and/or simulated annealing methods and/or methods which are based on genetic algorithms.

A system in accordance with claim 17 is specified as another means for achieving the object of the present invention. A particularly advantageous embodiment of this system is suitable for carrying out the method according to the invention.

Another embodiment of the system according to the invention exhibits an analytical facility for analysing the biopolymer to be investigated. For this purpose, the analytical facility is provided, for example, with analytical devices such as 2D PAGE robots, robots for punching out gel spots, protein digestion robots, MALDI sample preparation robots and the like which, according to one invention variant, are interlinked with each other.

In the case of the system, another embodiment envisages, for the classification according to the invention and/or for the further analysis, an evaluation facility which is based, for example, on a computer system and is also suitable, for example, for controlling the analytical devices and correspondingly automating the method according to the invention to the greatest extent possible.

Another embodiment of the system according to the invention particularly advantageously also envisages a database or a database interface.

According to another variant of the invention, the system exhibits visualization means which can be used, for example, to display analytical results and by means of which it is also possible to carry out an inter-active analysis where a user can alter their parameters during the analysis.

The implementation of the method according to the invention by means of a computer program in accordance with claims 23 and 24 is also of particular importance.

Other features, possible uses and advantages of the invention ensue from the following description of exemplary embodiments of the invention, which are depicted in the figures of the drawing.

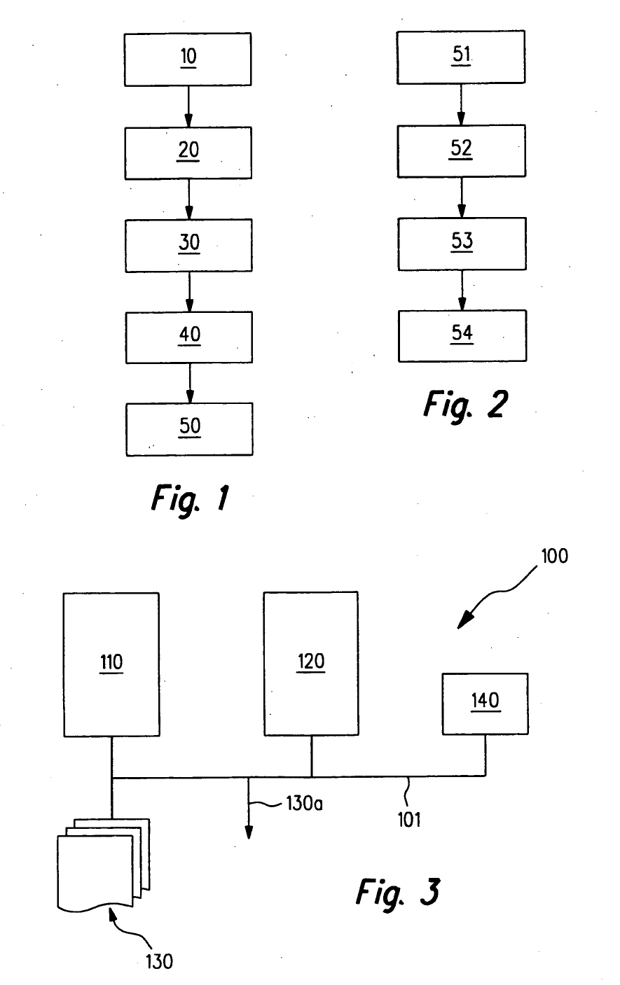

FIG. 1 diagrammatically shows a first embodiment of the method according to the invention in flow chart form,

FIG. 2 shows a flow chart which reproduces a stage of the method shown in FIG. 1 in detail,

FIG. 3 shows a block diagram of an embodiment of the system according to the invention,

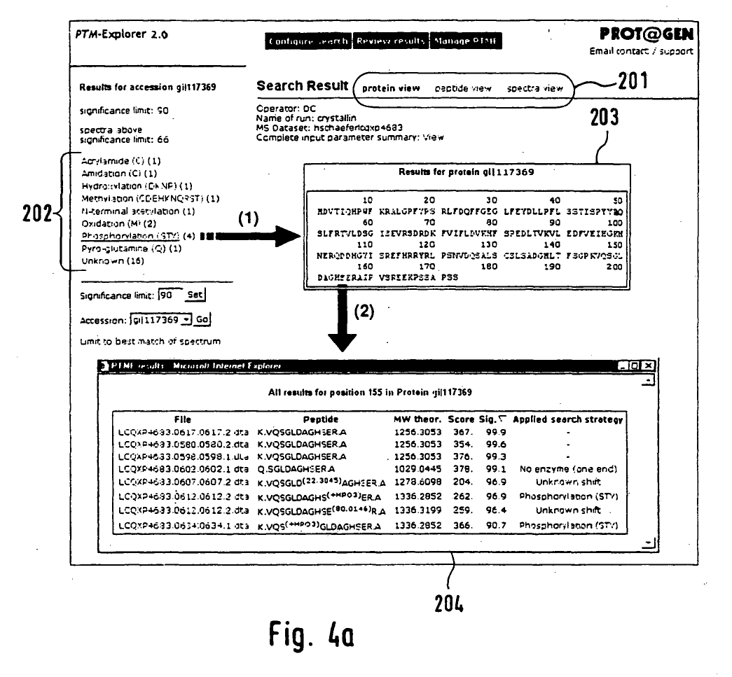

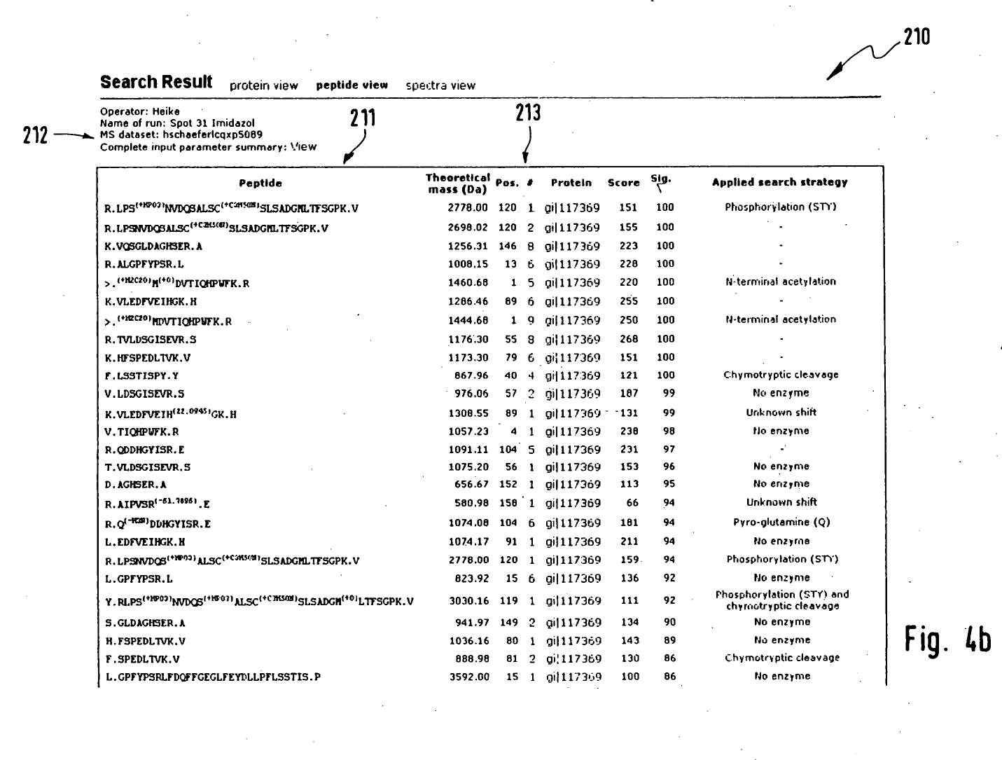

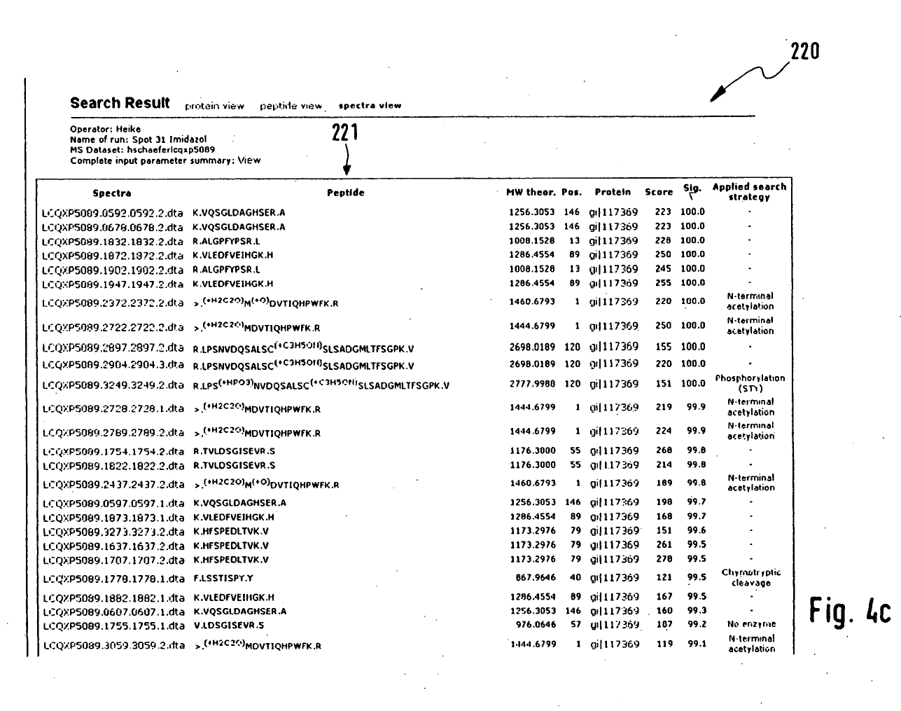

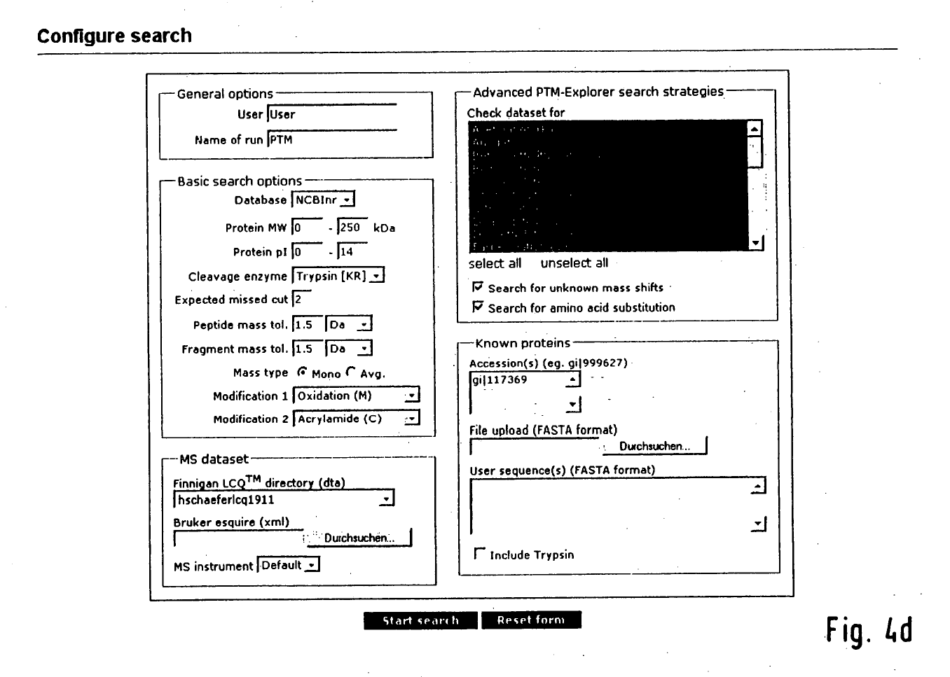

FIG. 4a shows a video display picture of an embodiment of the computer program according to the invention,

FIG. 4b shows another video display picture of an embodiment of the computer program according to the invention,

FIG. 4c shows a third video display picture of an embodiment of the computer program according to the invention, and

FIG. 4d shows a fourth video display picture of an embodiment of the computer program according to the invention.

In step 10 according to FIG. 1, a sample of a biopolymer to be investigated is first of all cleaved into fragments, with the cleavage being effected by the biopolymer sample being subjected to specific cleavage, for example by means of a known enzyme. The fragments which are obtained in this way are the peptides of which the biopolymer is composed.

A subsequent mass spectrometric analysis, in step 20, of the peptides which result from the cleavage of the biopolymer leads to mass spectra which give the molecular weight, and the relative quantity, of the peptides which have been obtained and which are therefore subsequently also described as being peptide mass spectra.

Using these peptide mass spectra, a primary structure of the biopolymer is determined in another step 30, in which a first sequence analysis is carried out. In this connection, the first sequence analysis 30 is effected in accordance with known methods, for example using a peptide mass fingerprint (PMF) algorithm or other known algorithms, or a combination of algorithms, which are not explained in more detail.

If, on the basis of other experimental data or on the basis of an experimental hypothesis, particular biopolymers whose sequences are partially or entirely known are suspected of being present in the investigated sample, the first sequence analysis in step 30 can also be used to assign, for the further investigation, these known biopolymer sequences to the measured mass spectra.

After that, the mass spectra are classified, in step 40, in dependence on the results of the first sequence analysis, cf. step 30, with at least one first and one second spectrum class being obtained. Those mass spectra to which it was possible to assign a known biopolymer within the context of the first sequence analysis 30 are assigned to the first spectrum class. That is, the first spectrum class contains those mass spectra which can be identified as being constituents of a known biopolymer.

Those mass spectra to which it was not possible to assign a known biopolymer within the context of the first sequence analysis 30 are assigned to the second spectrum class. This means that the second spectrum class contains those mass spectra whose appurtenant peptides could not yet be identified unambiguously as being constituents of a known biopolymer. These peptide mass spectra are also termed unidentified peptide mass spectra.

According to another variant of the method according to the invention, it is also possible to envisage more than two spectrum classes in order, for example, to be able to differentiate between the unidentified mass spectra with regard to characteristic properties. In this way, the total number of unidentified mass spectra can, for example, be divided up and a systematic further analysis, in which the unidentified mass spectra are processed, for example, in dependence on their characteristic properties, made possible. The classification of the mass spectra on the basis of their quality is an example in accordance with the invention. A suitable factor for assessing the quality of a mass spectrum can, for example, be obtained by means of an algorithm in dependence on the number and intensity of signals of the mass spectrum under consideration.

After the first sequence analysis 30 or the classification 40, the unidentified mass spectra, which are brought together in the second spectrum class, remain behind in the above-described method.

As can be seen from FIG. 1, the classification 40 is followed by a method step 50 which relates to a further analysis of the unidentified peptide mass spectra and whose further method steps 51 to 54 are specified in detail in the flow chart shown in FIG. 2.

What is termed a target sequence database (not shown), into which the known biopolymer, which was determined in the context of the first sequence analysis 30, is entered, is compiled at the beginning of the further analysis 50. Where several known biopolymers are present, each of the known biopolymers is entered into the target sequence database.

If biopolymers or biopolymer sequences are likewise known from an experiment hypothesis, they can also be added to the target sequence database. Thus, it is conceivable, for example, to insert trypsin into the target sequence database in connection with a tryptic cleavage of the analyzed biopolymer into peptides.

If known biopolymer sequences which were hypothetically present in the analyzed sample were obtained from further analyses, they can likewise be added to the target sequence database.

According to the invention, the further analysis 50 is then carried out in accordance with the method steps 51, 52, 53 and 54 which are described below. In this connection, all the steps 51 to 54 are preferably carried out separately for each biopolymer which is entered into the target sequence database.

In step 51, the known biopolymer from the target sequence database is modified on the basis of one or more modification rules, resulting in a modified biopolymer being obtained.

The modification rule specifies the way in which the known biopolymer is modified. For example, a modification rule which models a posttranslational modification of the known biopolymer comes into consideration in this connection.

The modified biopolymer is then cleaved, in step 52, in analogy with step 10, into fragments on the basis of one or more cleavage rules, with those peptides of which the modified biopolymer is composed being obtained as fragments in the present exemplary embodiment.

The cleavage rule specifies the way in which the given biopolymer from the target sequence database is cleaved. For example, the fact that the cleavage rule corresponds to the specificity of a protease enzyme which is used, or else the fact that the cleavage rule corresponds to an unspecific cleavage, comes into consideration in this connection.

Theoretical mass spectra are then formed in step 53. These theoretical mass spectra are obtained in dependence on the peptides of the modified biopolymer which are obtained in step 52.

Finally, step 54 provides for a comparison of the theoretical mass spectra formed in step 53 with the mass spectra of the fragments of the second spectrum class.

If it is possible to ascertain an adequate congruence of the theoretical mass spectra with the mass spectra of the second spectrum class it can then be assumed that the mass spectra of the second spectrum class can be assigned to a biopolymer which is present in the target sequence database or which only differs slightly, for example because of a modification, from a biopolymer in the target sequence database, as a result of which these mass spectra are no longer to be included in the unidentified peptide mass spectra. The results of this comparison can, for example, be quantified with weighting figures or with quality measurements which are, for example, obtained in dependence on a degree of congruence of investigated mass spectra.

The method steps 51 to 54 according to the invention, which are based on known biopolymers, can therefore be used to elucidate previously unidentified peptide mass spectra.

Investigations have shown that, as compared with conventional methods, up to 50% of the previously unidentified peptide mass spectra can be elucidated in this way.

In contrast to steps 10 and 20 in accordance with FIG. 1, steps 50 to 54 are not carried out on an available sample of the biopolymer but are, instead, only simulated, for example using a computer system which has been earmarked for this purpose.

Generally, the steps of modification and cleavage can be carried out in any order, i.e. the modification rule can be applied either before the cleavage rule or after the cleavage rule.

For example, the known biopolymer can, according to another variant of the invention, also initially be cleaved into fragments, preferably in accordance with a cleavage rule which can be preset. Subsequently, the fragments which have been obtained by the cleavage of the known biopolymer can be modified in accordance with a modification rule which can be preset. After that, theoretical mass spectra can be formed in dependence on the modified fragments, which mass spectra can then be compared with the mass spectra of the second spectrum class.

According to another very advantageous variant of the method according to the invention, the peptides can, in connection with the cleavage in accordance with step 10 in FIG. 1, also be cleaved, in an additional method step which is not depicted in FIG. 1, into peptide fragments, something which can be effected, for example, by impinging with impact gas in the mass spectrometer. The mass spectrometric analysis accordingly provides what are termed peptide fragment spectra, which can be analyzed, and compared with each other, in analogy with the peptide mass spectra. In particular, the method according to the invention is not restricted to evaluating only one category of mass spectra; it is also conceivable to investigate both peptide mass spectra and peptide fragment spectra and to correlate measurement results which are in each case obtained with each other.

Because of the greater accuracy, preference is given to using the combination of peptide mass spectra and peptide fragment spectra.

In another variant of the method according to the invention, the known biopolymer is cleaved on the basis of a cleavage rule which brings about an unspecific proteolysis of the known biopolymer from the target sequence database; the rule therefore acts, in particular, in method step 52 in FIG. 2. This results in the known biopolymer being cleaved, i.e. decomposed into peptides, at other sequence sites as compared with a specific, predetermined proteolysis. As a result, other theoretical mass spectra are formed in step 53.

After that, the theoretical mass spectra which have been formed in step 53 are in turn compared, in step 54, with the mass spectra of the fragments belonging to the second spectrum class.

If an adequate congruence of the theoretical mass spectra with the mass spectra of the fragments of the second spectrum class can be ascertained in the 54 comparison, it can then be assumed that the mass spectra of the appurtenant fragments of the second spectrum class are derived, by the above-described, modeled unspecific proteolysis, from the known biopolymer of the target sequence database. The number of unidentified mass spectra which remain can be reduced in this way as well.

In another variant of the method according to the invention, the known biopolymer is modified on the basis of a modification rule which models sequence errors, and another modification rule is provided for modeling amino acid substitutions. This makes it possible, in particular, to detect differences from primary structure information which is deposited in sequence databases and which is used for assigning the fragments or their mass spectrum to a biopolymer. In particular, differences caused by mutations can be elucidated in this way.

According to another, very advantageous embodiment of the invention, the known biopolymer is modified on the basis of another modification rule which models transpeptidations. In this connection, transpeptidation is understood as meaning the linking of a peptide bond of a cleavage product of a first peptide to an amino acid or to a second peptide when the first peptide is incubated with an enzyme in the presence of the second peptide or the amino acid.

Other modification rules are envisaged for modeling other possible modifications. The possible modifications can be taken, for example, from a modification database which contains known modifications and which may possibly also contain information about the given probability of occurrence, under predetermined conditions, of the modifications which are listed therein.

It is also possible to take account, in the case of the mass spectra or the known biopolymer, of modifications or mass differences which are not listed in a modification database. For this purpose, the theoretical total molecular weight is calculated for a suitable cleavage product of the known biopolymer and compared with an actual molecular weight which is determined from the mass spectra which are obtained by measurement, for example in step 20 (FIG. 1). A mass difference which may possibly ensue from this comparison is permuted to individual sequence positions, resulting in the formation of in each case new modified biopolymers which can be subjected to further analysis using the method according to the invention. This method is particularly suitable for elucidating peptide fragments or their mass spectra.

It is likewise possible to combine the modification rules and/or different cleavage rules.

In summary, the above-described process of modification and cleavage, cf. steps 51 and 52 in FIG. 2, or simply the selection of the modification rule(s) and/or cleavage rule(s), can be regarded as being the advancement of a hypothesis which states that previously unidentified peptide mass spectra or peptide fragment spectra are derived from the known biopolymer from the target sequence database as a result of the selected modification. This hypothesis is also termed a primary structure hypothesis.

The above-described procedure is not only used for elucidating the primary structure of the analyzed biopolymer; it can also make it possible to discover and characterize previously unknown types of biopolymer modifications or their combinations.

The method according to the invention can also be used for elucidating enzymic reactions or enzyme mechanisms since these frequently bring about the enzymic cleavage or modifications of biopolymers.

In connection with the modification in step 51 and also in connection with the cleavage in step 52, the primary structure hypothesis is examined or confirmed by means of forming the theoretic mass spectra and comparing them with mass spectra of the fragments of the second spectrum class in steps 53 and 54.

According to a particularly advantageous variant of the method, several or different modification rules and/or cleavage rules are combined in one primary structure hypothesis. It is also conceivable to advance a multistep system of primary structure hypotheses, with each primary structure hypothesis being based on one or more modification rules and/or cleavage rules.

Particularly advantageously, the modification rules and the cleavage rules or, respecively, the primary structure hypothesis(ses) are selected or, respectively, advanced in dependence on classified fragments, in particular in dependence on mass spectra of previously unidentified peptides or peptide fragments.

In another advantageous embodiment of the method according to the invention, known, preferably statistical optimization methods can be employed for selecting the modification rules and/or cleavage rules or for advancing the primary structure hypothesis(ses). It is particularly advantageous to use random walk methods and/or simulated annealing methods and/or methods which are based on genetic algorithms.

A system 100 according to the invention for elucidating the primary structure of biopolymers is depicted in simplified form in the block diagram shown in FIG. 3 and described below.

The system 100 possesses an analytical facility 110 which is suitable, in particular, for analysing the biopolymer to be investigated in accordance with the method steps 10, 20 and 30 which are depicted in FIG. 1. That is, the analytical facility 110 can be used to cleave a sample of the biopolymer to be investigated into fragments, i.e. into peptides or else into peptide fragments, something which, according to the above description, is effected, for example, by means of a specific digestion using an enzyme such as trypsin as well as using techniques such as PSD (post source decay) or CID (collision-induced decay).

The analytical facility 110 can also subject the fragments to a mass spectrometric analysis, cf. step 20 in FIG. 1, resulting in peptide mass spectra or peptide fragment spectra being obtained.

The peptide mass spectra or the peptide fragment spectra can then be supplied to a first sequence analysis 30 which is likewise carried out using the analytical facility 110.

In the system 100, the data which are obtained in the first sequence analysis 30 are transferred, by way of a data bus 101, to an evaluation facility 120, which carries out a classification in accordance with step 40 in FIG. 1 and the further analysis in accordance with step 50.

For example, the evaluation facility 120 is configured as a computer system which can, inter alia, also control the analytical facility 110, which usually comprises a large number of different analytical devices (not shown). The analytical devices comprise, for example, 2D PAGE robots, robots for punching out gel spots, protein digestion robots, MALDI sample preparation robots and the like.

It is likewise conceivable for the individual analytical devices to be interlinked with each other by means of a wire-linked or wireless data bus and/or control bus or connected using the data bus 101.

For the purpose of carrying out the first sequence analysis 30, FIG. 1, and/or the subsequent analysis 50, it is provided for the system 100 to have a database linkage such that the analytical facility 110 and/or the evaluation facility 120 is/are able to access databases 130 by way of the data bus 101. These databases 130 can be present locally at the site of the system 100 or else be effected on a computer system or the like which is interlinked using the data bus 101. Finally, it is also possible for the databases 130 to be dispersed databases which are, for example, effected by means of a composite of computer systems which are interlinked with each other, with it also being possible, for example, for this composite to be linked to the internet.

The databases 130 are, for example, a sequence database which contains the amino acid sequences of known biopolymers and, where appropriate, other data regarding the given biopolymers. Such a database is used in the context of the first sequence analysis 30 as well as, for example, in step 54 (FIG. 2), see above.

The databases 130 can also contain, or constitute, modification databases which contain information regarding different modifications or modification rules which are used in the method according to the invention, in particular in steps 51 and 52.

Furthermore, the databases 130 are also envisaged, in accordance with the invention, for effecting the already described target sequence database into which the known biopolymer(s), which was/were determined in the context of the first sequence analysis 30, is/are entered.

A database interface 130a, by way of which the system 100 can be connected to other databases (not depicted), is likewise envisaged in the system 100. For example, it is possible, in this way, for unidentified fragments or their mass spectra to be exchanged with other systems 100.

Particularly advantageously, the system 100 is equipped with visualization means 140 which make it possible to visualize status messages and/or analytical results of the system 100 and the like. This thereby at the same time gives a user of the system 100 the possibility of configuring the system 100 or its components and, for example, specifying parameters for the method steps 10 to 50 and 51 to 54.

In a very advantageous variant of the system 100 according to the invention, the visualization means 140 are formed by a computer system and a corresponding indicating device such as a monitor, with a user surface which is preferably window-oriented, and which enables the system 100 to be operated comfortably and efficiently, preferably being envisaged. In this connection, the user surface is part of a computer program which is suitable for implementing the method according to the invention and also for actuating the system 100 or its components.

FIG. 4a shows a video display picture of the user surface according to the invention in which different ways of depicting analytical results can be selected in a region 201. As can be seen from FIG. 4a, this region provides for a “spectra view” visualization of the mass spectra, a “peptide view” visualization of the peptides or peptide fragments which have been determined and a “protein view” protein-related visualization.

A region 202 which is arranged on the left-hand side in FIG. 4a lists different modifications which the user can in each case select. In the present case, the modification selected is a “phosphorylation (STY)” phosphorylation. A mouse click on this phosphorylation displays, in a separate display panel 203 which is provided for this purpose, all the amino acids which exhibit the phosphorylation. This process is symbolized by the arrow 1 in FIG. 4a.

The user can likewise select the amino acids which are displayed in the display panel 203 by means of a mouse click whereupon all the mass spectra in which a corresponding sequence position is present are displayed in another display panel 204.

In one embodiment of the present invention, the visualization of peptides which has already been described in connection with the video display picture in FIG. 4a is effected using the video display picture 210 which is depicted in FIG. 4b and in which the displayed peptides are listed in tabular form in a first column 211. In all, the video display picture 210 lists, for example, all the peptides which it was possible to determine using a particular number of mass spectra. In this connection, this number of mass spectra is advantageously combined in what is termed a spectral data set whose name is listed at the place on the video display picture which is indicated with the reference number 212.

If several mass spectra are to lead to the same peptide being determined, the peptide concerned is only cited once in the list in the video display picture 210. In this case, the column 213 marked with a diesis shows how many mass spectra lead or point to the same peptide. A mouse click on the given numerical value in column 213 results in the relevant mass spectra being displayed, with the display preferably taking place in a separate window or in a separate region of the video display picture 210 which is envisaged for this purpose.

The indication, which is explained with the aid of the video display picture 210, of the peptides is particularly advantageous for the evaluation or verification of the ascertained data by a user, who can, with little effort, display all the mass spectra which point to the given peptides.

The video display picture 220, which is also designated “spectra view”, in FIG. 4c gives a tabular listing of all the mass spectra as well as, in column 221, a peptide which has been determined for the given mass spectrum and which has, for example, been found using the method according to the invention. It is particularly expedient to depict ascertained data in accordance with FIG. 4c when an elucidation of a particular mass spectrum is of interest.

The video display picture shown in FIG. 4d is an input picture for parameters which can be used for controlling the method according to the invention or system 100 (FIG. 3).

Generally, the method according to the invention and system 100 also make it possible to analyse a protein mixture containing several proteins, for example, as well as only one single biopolymer.

The method according to the invention is furthermore suitable for obtaining information with regard to previously unknown modifications of biopolymers or previously unknown cleavages. The biopolymer to be investigated which is used for this purpose is a biopolymer whose primary structure is already elucidated, i.e. known. It is possible, from an analysis of the mass spectra, which are obtained in the method according to the invention, of peptides and/or peptide fragments of this biopolymer, to evaluate, for example, differences between the mass spectra which are obtained analytically and the known mass spectra of the biopolymer in order, from this, to identify previously unknown modifications or cleavages.

In this way, it is also possible to use the method according to the invention and system to elucidate the mechanisms on which the modification or cleavage is based.

According to another very advantageous embodiment of the method according to the invention, the above-described process of the further analysis 50 can also be carried out without the modification step 51 and/or the cleavage step 52.

This thereby takes into account, inter alia, the fact that the biopolymer can be cleaved without any previous modification. For example, an unclarified mass spectrum can also arise from the known biopolymer simply due to one or more unexpected cleavages. In this case, it is advantageous, before forming the theoretical mass spectra in step 53, to carry out only one cleavage without previously modifying the biopolymer.

The modification 51 prior to the cleavage 52 can therefore be dispensed with, under certain circumstances. It is likewise possible for a cleavage 52 of a modified biopolymer to be dispensed with, where appropriate.

Since it is possible, up to a certain molecular weight, to acquire mass spectra of whole proteins directly, it is also possible to directly compare mass spectra which have been obtained in this way with theoretical mass spectra which have been obtained in accordance with the invention, with it being possible to identify a particular modification when the mass spectrum congruence is adequate. In this case, there is no need for the cleavage step 52.

1. A method for elucidating the primary structure of biopolymers, in which a biopolymer to be investigated is cleaved into fragments and, after that, subjected to a mass spectrometric analysis (20), resulting in mass spectra being obtained, and in which known algorithms are used for a first sequence analysis (30) of the fragments in order to determine a primary structure of the biopolymer using the mass spectra, wherein the mass spectra are classified in dependence on results of the first sequence analysis (30), resulting in at least one first spectrum class, to which a known biopolymer can be assigned, and one second spectrum class, to which no known biopolymer can be assigned, being obtained, and in that a further analysis (50) of mass spectra of the second spectrum class is carried out in dependence on the known biopolymer.

2. The method as claimed in claim 1, wherein the known algorithms used for the first sequence analysis (30) and/or the further analysis (50) are a peptide mass fingerprint (PMF) algorithm and/or a peptide fragmentation fingerprint (PFF) algorithm and/or algorithms from the family of the de-novo sequencing algorithms and/or PTM prediction algorithms and/or comparable algorithms.

3. The method as claimed in claim 1, wherein the further analysis (50) exhibits the following steps:
modifying (51) the known biopolymer in accordance with a modification rule which can be preset in order to obtain a modified biopolymer,
cleaving (52) the modified biopolymer into fragments, preferably in accordance with a cleavage rule which can be preset,
forming (53) theoretical mass spectra in dependence on the fragments which are obtained in connection with the cleaving (52) of the modified biopolymer,
comparing (54) the theoretical mass spectra with the mass spectra of the second spectrum class.

4. The method as claimed in claim 1, wherein the further analysis (50) exhibits the following steps:
cleaving the known biopolymer into fragments, preferably in accordance with a cleavage rule which can be preset,
modifying the fragments, which have been obtained by the cleavage of the known biopolymer, in accordance with a modification rule which can be preset in order to obtain modified fragments,
forming theoretical mass spectra in dependence on the modified fragments,
comparing (54) the theoretical mass spectra with the mass spectra of the second spectrum class.

5. The method as claimed in claim 3, wherein use is made, for the modification (51) of a modification rule by means of which
a. a posttranslational modification and/or
b. an amino acid substitution and/or
c. a sequence error and/or
d. a transpeptidation and/or
e. random and/or
f. other modifications
of the known biopolymer can be modeled.

6. The method as claimed in claim 3, wherein use is made, for the cleavage, of a cleavage rule by means of which specific and/or unspecific cleavages of the known biopolymer and/or of the modified biopolymer can be modeled, with the cleavage rule preferably being determined in dependence on data from a cleavage database.

7. The method as claimed in claim 3, wherein the steps of modification (51) and of cleavage (52) can be used in any order and/or several times and/or in that the cleavage step (52) and/or the modification step (51) is dispensed with.

8. The method as claimed in claim 3, wherein the modification rule is formed in dependence on data from a modification database (130).

9. The method as claimed in claim 1, wherein peptides are obtained, as fragments of the biopolymer, in connection with the cleavage (10) of the biopolymer to be investigated.

10. The method as claimed in claim 1, wherein peptide fragments are obtained, as fragments of the biopolymer, in connection with the cleavage (10) of the biopolymer to be investigated.

11. The method as claimed in claim 1, wherein several known algorithms are combined for the sequence analysis in connection with the first sequence analysis (30) and/or in connection with the further analysis (50).

12. The method as claimed in claim 1, wherein single-step or multi-step primary structure hypotheses are advanced for the further analysis (50) of mass spectra which are preferably of the second spectrum class.

13. The method as claimed in claim 12, wherein the advancement of the primary structure hypotheses comprises the selection of modification rules by means of which
a. a posttranslational modification and/or
b. an amino acid substitution and/or
c. a sequence error and/or
d. a transpeptidation and/or
e. random and/or
f. other modifications
of the known biopolymer can be modeled.

14. The method as claimed in claim 12, wherein the advancement of the primary structure hypotheses comprises the advancement of cleavage rules by means of which specific and/or unspecific cleavages can be modeled.

15. The method as claimed in claim 12, wherein the primary structure hypotheses are advanced in dependence on mass spectra which are preferably of the second spectrum class.

16. The method as claimed in claim 12, wherein the advancement of the primary structure hypotheses is effected using statistical optimization methods, in particular.

17. A system (100) for elucidating the primary structure of biopolymers, in which a biopolymer to be investigated can be cleaved into fragments and, after that, supplied to a mass spectrometric analysis (20), resulting in mass spectra being obtained, and in which known algorithms can be used for a first sequence analysis (30) of the fragments in order to determine a primary structure of the biopolymer using the mass spectra, wherein the mass spectra can be classified in dependence on results of the first sequence analysis (30), resulting in at least one first spectrum class, to which a known biopolymer can be assigned, and one second spectrum class, to which no known biopolymer can be assigned, being obtained, and in that a further analysis (50) of mass spectra of the second spectrum class can be carried out in dependence on the known biopolymer.

18. The system (100) as claimed in claim 17, wherein the system (100) is suitable for implementing the method as claimed in claim 1.

19. The system (100) as claimed in claim 17, wherein the system (100) exhibits an analytical facility (110) for analysing the biopolymer to be investigated.

20. The system (100) as claimed in claim 17, wherein the system (100) exhibits an evaluation facility (120), in particular for the classification (40) and/or for the further analysis (50).

21. The system (100) as claimed in claim 17, wherein the system (100) exhibits at least one database (130) and/or one database interface (130a).

22. The system (100) as claimed in claim 17, wherein the system (100) exhibits visualization means (140).

23. A computer program for controlling the system (100) as claimed in claim 17.

24. The computer program as claimed in claim 23, wherein the computer program is suitable for implementing the method of claim 1.
